(19) United States
(12) Patent Application Publication
(10) Pub. No.: US 2006/0052620 A1
(43) Pub. Date: Mar. 9, 2006

(54) Preparation of substituted indenes

(76) Inventors: Jorg L. Schulte, Frankfurt (DE); Carsten Bingel, Schifferstadt (DE); Jorg Schottek, Frankfurt (DE)

Correspondence Address: NOVAK DRUCE DELUCA & QUIGG, LLP, 1300 EYE STREET NW, SUITE 400 EAST, WASHINGTON, DC, 20005, US

(21) Appl. No.: 10/509,751

(22) PCT Filed: Apr. 5, 2003
(86) PCT No.: PCT/EP03/03562

§ 371 Date: May 19, 2005

Related U.S. Application Data
(60) Provisional application No. 60/399,761, filed on Jul. 31, 2002.

(30) Foreign Application Priority Data
Apr. 10, 2002 (DE) 102 15 849.5

(51) Int. Cl. C07F 17/00; C07C 13/465

(52) U.S. Cl. 556/43; 556/46; 585/27

(57) ABSTRACT

The present invention relates to a simple process for preparing alkyl-substituted indenes of the formula (II) in improved yields from indenes of the formula (I) by reaction with compounds of the formula (III), to the alkyl-substituted indenes prepared in this way and to the use of these for the preparation of metallocenes for highly active polymerization catalysts.

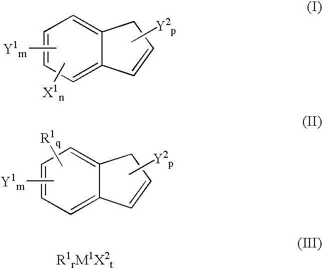

The present invention relates to a simple process for preparing alkyl-substituted indenes in improved yields, to the alkyl-substituted indenes prepared in this way and to their use for preparing metallocenes for highly active polymerization catalysts.

Substituted indenes are important intermediates for preparing active compounds in the fields of pharmacy (Negwer, VCH 1987, p. 1703 ff.), crop protection, fine chemicals, liquid crystals and metallocene catalysts for the polymerization of α-olefins (Chem. Rev. 2000, Number 4). Use of substituted indenes as starting materials makes it possible to obtain chiral ansa-metallocenes which are of great importance as transition metal components of highly active catalysts in the stereospecific polymerization of olefins (cf. EP 0 129 368).

Variation of the ligand system, for example by means of substitution, enables the catalyst properties to be influenced in a targeted manner. This makes it possible to alter the polymer yield, the molecular weight distribution, the tacticity and the melting point of the polymers to the desired degree (Chem. Rev. 2000, Number 4). Bridged zirconocenes containing, as n ligands, indenyl radicals which bear the bridge in position 1, preferably bear a hydrocarbon radical in position 2 and bear a hydrocarbon radical in position 4 have been found to be particularly highly active and stereoselective catalyst systems (EP 0 567 970 A1; EP 0 629 632 A2). The ligand systems used for these highly active metallocenes are prepared from the corresponding indenes.

A number of processes comprising an inexpensive coupling reaction have been described for the preparation of aryl-substituted indenes (WO 98/40331; U.S. Pat. No. 5,789,634). However, the synthesis of alkyl-substituted indenes is difficult since the alkyl substituents either have to be present in the starting material at the commencement of the synthesis, for example in the case of complicated ring closure syntheses (cf., for example, J. Organomet. Chem. 1995, 181-194) or, in the case of coupling reactions, comparatively expensive but sufficiently reactive bromoindenes are used as starting material (cf., for example, J. Org. Chem. 1984, 49, 4226-4237), while such coupling reactions of chloroindenes cannot be carried out or give unsatisfactory yields because of a lack of reactivity.

It is an object of the present invention to find a simple, flexible, inexpensive process for preparing substituted indenes which are important intermediates for the preparation of active compounds and metallocene complexes.

We have found that this object is achieved by conversion of substituted indenes containing chlorine as leaving group in the six-membered ring of the indene in a simple manner into substituted indenes which are bound via an sp3 center and can be used, inter alia, for the preparation of active compounds and metallocene complexes.

The present invention accordingly provides a process for preparing indenes of the formula (II) from indenes of the formula (I) via a transition metal-catalyzed sp2-sp3 coupling reaction of aryl chlorides with aliphatic organometallic compounds.

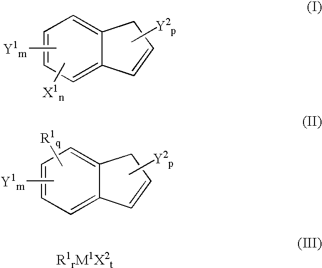

Here, an indene of the formula (I) is reacted with a coupling component of the formula (III) in a suitable solvent with addition of a suitable transition metal catalyst to form an indene of the formula (II), where, in the formulae (I), (II) and (III),
X¹ is chlorine;
Y¹ are identical or different and are selected independently from the group consisting of C₁-C₄₀ groups, e.g. C₁-C₂₅-alkyl, C₂-C₂₅-alkenyl, C₂-C₂₅-alkynyl, C₃-C₁₅-alkylalkenyl, C₃-C₁₅-alkylalkynyl, C₆-C₂₄-aryl, C₄-C₂₄-heteroaryl, C₅-C₂₄-alkylheteroaryl, C₇-C₃₀-arylalkyl, C₇-C₃₀-alkylaryl, C₁-C₁₂-alkoxy, C₆-C₂₄-aryloxy, fluorinated C₁-C₂₅-alkyl, fluorinated C₆-C₂₄-aryl, fluorinated C₇-C₃₀-arylalkyl, fluorinated C₇-C₃₀-alkylaryl, and the fluorine atom and heteroatom-containing groups, e.g. boron-, silicon-, nitrogen-, oxygen- or sulfur-containing groups, which may bear one or more substituents, where a plurality of groups Y¹ may also together form a cyclic aliphatic or aromatic ring system which may in turn be substituted and may contain heteroatoms;
Y² are identical or different and are selected independently from the group consisting of C₁-C₄₀ groups, e.g. C₁-C₂₅-alkyl, C₂-C₂₅-alkenyl, C₂-C₂₅-alkynyl, C₃-C₁₅-alkylalkenyl, C₃-C₁₅-alkylalkynyl, C₆-C₂₄-aryl, C₄-C₂₄-heteroaryl, C₅-C₂₄-alkylheteroaryl, C₇-C₃₀-arylalkyl, C₇-C₃₀-alkylaryl, C₁-C₁₂-alkoxy, C₆-C₂₄-aryloxy, fluorinated C₁-C₂₅-alkyl, fluorinated C₆-C₂₄-aryl, fluorinated C₇-C₃₀-arylalkyl, fluorinated C₇-C₃₀-alkylaryl, and heteroatom-containing groups, e.g. boron-, silicon-, nitrogen-, oxygen- or sulfur-containing groups, which may bear one or more substituents, where a plurality of groups y² may also together form a cyclic aliphatic or aromatic ring system which may in turn be substituted and may contain heteroatoms;
R¹ are identical or different and are selected independently from the group consisting of linear, branched or cyclic aliphatic hydrocarbon groups, e.g. C₁-C₂₅-alkyl which may in turn bear a variety of substituents, and groups bound via an aliphatic group to the indenyl skeleton, e.g. C₃-C₁₅-alkenylalkyl, C₃-C₁₅-alkynylalkyl, C₅-C₂₄-heteroarylalkyl, C₇-C₃₀-arylalkyl, C₂-C₃₀-alkyloxyalkyl, C₇-C₃₀-aryloxyalkyl, C₈-C₃₀-alkylarylalkyl, and other heteroatom-containing groups which are bound via an aliphatic group to the indenyl skeleton, e.g. boron-, silicon-, nitrogen-, oxygen- or sulfur-containing groups, and may bear one or more substituents;
M¹ is an element of group 1, 2, 12, 13 or 14 of the Periodic Table of the Elements, preferably Li, Na, K, Rb, Cs, Be, Mg, Ca, Sr, Ba, B, Sn, Zn, Cd or Hg, particularly preferably Li, Na, K, Mg, Ca, B, Sn, Zn, Cd or Hg, very particularly preferably Li, Mg, B or Zn;
X² are identical or different and are selected independently from the group consisting of halogen atoms, the hydroxy group, alkoxy groups, aryloxy groups, mesylate, tosylate and triflate, preferably from the group consisting of halogen atoms, the hydroxy group, alkoxy and aryloxy;
m is an integer from 0 to 3;
n is an integer from 1 to 4;
p is an integer from 0 to 4;
q is an integer from 1 to 4;
r is 1, 2 or 3, and
t is 0, 1 or 2, where r+t corresponds to the oxidation number of M¹.

In the process of the present invention, particular preference is given to reacting an indene of the formula (i) with a coupling component of the formula (III) to form an indene of the formula (II), where, in the formulae I, II and III,
M¹ is Li, Mg, B or Zn, in particular Mg;
X² are identical or different and are selected independently from the group consisting of halogen atoms, the hydroxy group, alkoxy groups and aryloxy groups, in particular from the group consisting of halogen atoms such as chlorine, bromine and iodine; and the remaining radicals and indices are as defined above.

The positions of the substituents on the indene skeleton are numbered as follows:

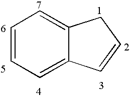

For the purposes of the present invention, alkyl refers to linear, branched or cyclic alkyl radicals such as methyl, ethyl, n-propyl, isopropyl, n-butyl, sec-butyl, isobutyl, tert-butyl, n-pentyl, cyclopentyl, n-hexyl or cyclohexyl.

In the process of the present invention, particular preference is given to indenes of the formula (I) in which n, namely the number of leaving groups X¹, is 1 or 2, preferably 1,
m is 0 or 1, in particular 0,
Y¹ is C₁-C₁₀-alkyl, C₆-C₁₀-aryl or C₇-C₁₄-alkylaryl,
p is 0 or 1, preferably 1, and
Y² is C₁-C₁₀-alkyl, C₆-C₁₀-aryl or C₇-C₁₄-alkylaryl, in particular C₁-C₆-alkyl, and Y² is preferably a substituent in position 2 of the indene skeleton.

R¹ is preferably a linear, branched or cyclic C₁-C₁₈-alkyl group, in particular C₁-C₁₀-alkyl group, a C₇-C₁₄-arylalkyl group or a C₈-C₁₅-alkylarylalkyl group.

q is preferably 1 or 2, in particular 1.

Illustratrative examples of indenes of the formula (I) as starting materials for the process of the present invention, which do not, however, restrict the scope of the invention, are: 4-chloro-1-indene; 5-chloro-1-indene; 6-chloro-1-indene; 7-chloro-1-indene; 2-methyl-4-chloro-1-indene; 2,7-dimethyl-4-chloro-1-indene; 2,4-dimethyl-7-chloro-1-indene; 2-methyl-5-chloro-1-indene; 2-methyl-6-chloro-1-indene; 2-methyl-7-chloro-1-indene; 2-ethyl-4-chloro-1-indene; 2-ethyl-5-chloro-1-indene; 2-ethyl-6-chloro-1-indene; 2-ethyl-7-chloro-1-indene; 2-propyl-4-chloro-1-indene; 2-propyl-5-chloro-1-indene; 2-propyl-6-chloro-1-indene; 2-propyl-7-chloro-1-indene; 2-i-propyl-4-chloro-1-indene; 2-i-propyl-5-chloro-1-indene; 2-i-propyl-6-chloro-1-indene; 2-i-propyl-7-chloro-1-indene; 2-butyl-4-chloro-1-indene; 2-butyl-5-chloro-1-indene; 2-butyl-6-chloro-1-indene; 2-butyl-7-chloro-1-indene; 2-s-butyl-4-chloro-1-indene; 2-s-butyl-5-chloro-1-indene; 2-s-butyl-6-chloro-1-indene; 2-s-butyl-7-chloro-1-indene; 2-t-butyl-4-chloro-1-indene; 2-t-butyl-5-chloro-1-indene; 2-t-butyl-6-chloroindene; 2-t-butyl-7-chloroindene.

The starting materials of the formula (I) used in the synthetic process of the present invention can be synthesized in a simple manner by customary methods of the prior art. Thus, for example, 4-chloro-2-alkylindenes of the formula (I) can be prepared from the corresponding 7-chloro-2-alkyl-1-indanones by hydrogenation of the carbonyl group to an alcohol group (indanol) by means of suitable hydrides, for example sodium borohydride, and subsequent elimination of water, in particular under acid conditions, for example using para-toluenesulfonic acid. The 4-chloro-2-alkyl-1-indanones used as starting materials are prepared by methods of the prior art described in WO 98/40331. The isomeric 7-chloro-2-alkylindenes can be prepared, for example, as described in U.S. Pat. No. 5,789,634.

The aliphatic organometallic compounds of the formula (III) are likewise obtainable in a simple manner by standard methods of the prior art or can be purchased commercially. The synthesis of appropriate Grignard reagents is described, for example, in Holm, Torkil, J. Chem. Soc. Perkin Trans. 2, 1981, 464-467. The synthesis of further organometallic reagents of the formula (III) involves standard methods of organometallic chemistry and is described, for example, in March, Advanced Organic Chemistry, 4th Edition 1992, and the references cited therein.

The synthesis according to the present invention of the indenes of the formula (II) which are substituted by alkyl groups in the six-membered ring is carried out by means of a transition metal-catalyzed sp²-sp³ coupling reaction of chloroindenes of the formula (I) with aliphatic organometallic compounds of the formula (III). These reactions are carried out in suitable solvents, for example diethyl ether, tetrahydrofuran, etc., under a protective gas atmosphere.

As transition metal catalysts, it is in principle possible to use transition metal complexes of groups 8 to 10 of the Periodic Table of the Elements, in particular group 10. Particularly useful complexes are complexes of nickel or palladium, in particular complexes of nickel, for example [1,3-bis(diphenylphosphino)propane]nickel(II) chloride (hereinafter referred to as NiCl₂[dppp]₂). Illustratrative examples of transition metal catalysts which do not, however, restrict the scope of the invention are nickel(II) acetylacetonate, [1,2-bis(diphenylphosphino)ethane]nickel(II) chloride (hereinafter referred to as NiCl₂[dppe]₂), [1,1′-bis(diphenylphosphino)ferrocene]nickel(II) chloride (hereinafter referred to as NiCl₂[dppf]₂), bis(tributylphosphine)nickel(II) bromide, bis(tributylphosphine)nickel(II) chloride, bis(triphenylphosphine)nickel(II) chloride, bis(triphenylphosphine)dicarbonylnickel(0), [1,2-bis(dimethylphosphino)ethane]nickel(II) chloride, bis(triethylphosphine)nickel(II) chloride, bis(triphenylphosphine)palladium(II) chloride, tetrakis(triphenylphosphine)palladium(0), [1,2-bis(diphenylphosphino)ethane]palladium(II) chloride and the [1,1′-bis(diphenylphosphino)ferrocene]nickel(II) chloride-methylene chloride complex.

The nickel complexes NiCl₂[dppp]₂, NiCl₂[dppe]₂, NiCl₂[dppf]₂ and NiCl₂[PPh₃]₂ are particularly useful. The use of NiCl₂[dppp]₂ is especially advantageous.

Corresponding reactions have hitherto been described by Kumada et al. as cross-coupling reactions using chlorobenzene or dichlorobenzene and Grignard reagents (cf., for example, K. Tamahu, K. Sumidani, M. Kumada, J. American Chemical Soc. 1972, 94, 4374), but not using chloroindenes having an acidic proton which can be abstracted by means of strong bases such as butyl-lithium, potassium hydride, methyllithium or dibutylmagnesium.

Illustrative but nonrestrictive examples of aliphatic or organometallic compounds of the formula (III) apart from aliphatic Grignard compounds are, for example, methyllithium, n-butyllithium, di-butylmagnesium, methylboronic acid, ethylboronic acid, trimethylaluminum, ethylaluminum dichloride, methylzinc chloride and dimethylzinc.

Examples of the aliphatic Grignard compounds of the formula (III) which are preferred in the process of the present invention are methylmagnesium chloride, methylmagnesium bromide, methylmagnesium iodide, ethylmagnesium chloride, ethylmagnesium bromide, 2-phenylethylmagnesium chloride, n-propylmagnesium chloride, n-butylmagnesium bromide, hexylmagnesium bromide, benzylmagnesium chloride, 4-methylbenzylmagnesium chloride, cyclohexylmethylmagnesium bromide, (4′-1-propylphenyl)methylmagnesium chloride, (4′-t-butylphenyl)methylmagnesium chloride, isopropylmagnesium chloride, sec-butylmagnesium chloride, cyclohexylmagnesium bromide, exo-2-norbornylmagnesium bromide and tert-butylmagnesium chloride. Particular preference is given to bulky aliphatic Grignard compounds of the benzyl type and ones in which the magnesium is bound to a tertiary carbon atom, for example as in cyclohexyl-magnesium bromide, or to a quaternary carbon atom, for example as in tert-butylmagnesium chloride, in particular to a tertiary carbon atom.

In most cases it is sufficient to use the particularly preferred catalyst NiCl₂[dppp]₂ in an amount of from 0.01 to 5 mol %, preferably from 1 to 3 mol % and particularly preferably about 2 mol %, in each case based on the chloroindene of the formula (I) used.

Suitable solvents for the nickel-catalyzed cross-coupling of chloroindenes of the formula (I) with aliphatic organometallic compounds of the formula (III) are, for example, aliphatic ethers such as diethyl ether, dibutyl ether, methyl tert-butyl ether, tetrahydrofuran and dioxane, aliphatic hydrocarbons such as pentane, hexane and the like, aromatic hydrocarbons such as benzene, toluene, xylenes and the like. Mixtures of various solvents in various mixing ratios can also be used according to the present invention to match the solubility of the starting materials and end products to the reaction conditions in an optimal fashion.

A person skilled in the art will choose solvents or solvent mixtures appropriate to the specific substitution pattern of the starting compounds and the catalysts and reagents used by means of simple tests or on the basis of known solubilities and solvent properties.

In preferred embodiments of the process of the present invention, the chloroindene of the formula (I) together with a sufficient amount of solvent are placed in a reaction vessel under protective gas, the catalyst is subsequently added and a solution of the aliphatic organometallic compound of the formula (III) in a suitable solvent is then slowly added dropwise at low temperature, preferably in the range from −78° C. to +10° C. It is likewise possible to place the aliphatic organometallic compound III together with the catalyst in the reaction vessel and then to slowly add the chloroindene compound of the formula (I) in a suitable solvent dropwise. A person skilled in the art will choose an appropriate procedure depending on the reactivity and substitution of the individual reagents.

Preferred examples of substituted indenes of the formula (II) which can be prepared according to the present invention are, without being restricted to these:
2,7-dimethyl-1-indene, 2,7-diethyl-1-indene, 2,7-di-n-propyl-1-indene, 2,7-diisopropyl-1-indene, 2,4-dimethyl-1-indene, 2,4-diisopropyl-1-indene,
7-methyl-2-ethyl-1-indene, 4-methyl-2-ethyl-1-indene, 7-ethyl-2-methyl-1-indene, 7-methyl-2-n-propyl-1-indene, 7-ethyl-2-n-propyl-1-indene, 7-n-propyl-2-methyl-1-indene, 7-n-propyl-2-ethyl-1-indene, 7-methyl-2-i-propyl-1-indene, 4-methyl-2-i-propyl-1-indene, 7-ethyl-2-i-propyl-1-indene, 7-n-propyl-2-i-propyl-1-indene, 7-i-propyl-2-methyl-1-indene, 7-i-propyl-2-ethyl-1-indene, 7-i-propyl-2-n-propyl-1-indene,
7-cyclohexyl-2-methyl-1-indene, 4-cyclohexyl-2-methyl-1-indene, 7-cyclohexyl-2-ethyl-1-indene, 7-cyclohexyl-2-n-propyl-1-indene, 7-cyclohexyl-2-i-propyl-1-indene, 4-cyclohexyl-2-i-propyl-1-indene, 7-cyclohexylmethyl-2-methyl-1-indene, 4-cyclohexylmethyl-2-methyl-1-indene, 7-cyclohexylmethyl-2-ethyl-1-indene, 7-cyclohexylmethyl-2-n-propyl-1-indene, 7-cyclohexylmethyl-2-i-propyl-1-indene, 4-cyclohexylmethyl-2-i-propyl-1-indene, 7-(2′-adamantyl)-2-methyl-1-indene, 4-(2′-adamantyl)-2-methyl-1-indene, 7-(2′-adamantyl)-2-ethyl-1-indene, 7-(2′-adamantyl)-2-i-propyl-1-indene, 4-(2′-adamantyl)-2-i-propyl-1-indene, 7-(exo-2-norbornyl)-2-methyl-1-indene, 4-(exo-2-norbornyl)-2-methyl-1-indene, 7-(exo-2-norbornyl)-2-ethyl-1-indene, 7-(exo-2-norbornyl)-2-i-propyl-1-indene, 4-(exo-2-norbornyl)-2-i-propyl-1-indene, 7-benzyl-2-methyl-1-indene, 4-benzyl-2-methyl-1-indene, 4-benzyl-2-ethyl-1-indene, 7-benzyl-n-propyl-1-indene, 7-benzyl-2-i-propyl-1-indene, 4-benzyl-2-i-propyl-1-indene, 7-(1′-adamantylmethyl)-2-methyl-1-indene, 4-(1′-adamantylmethyl)-2-methyl-1-indene, 7-(1′-adamantylmethyl)-2-ethyl-1-indene, 7-(1′-adamantylmethyl)-2-i-propyl-1-indene, 4-(1′-adamantylmethyl)-2-i-propyl-1-indene,
7-[(4′-i-propylphenyl)methyl]-2-methyl-1-indene, 7-[(4′-i-propylphenyl)methyl]-2-ethyl-1-indene, 7-[(4′-i-propylphenyl)methyl]-2-n-propyl-1-indene, 7-[(4′-i-propylphenyl)methyl]-2-i-propyl-1-indene, 4-[(4′-t-butylphenyl)methyl]-2-methyl-1-indene, 4-[(4′-t-butylphenyl)methyl]-2-ethyl-1-indene, 4-[(4′-t-butylphenyl)methyl]-2-n-propyl-1-indene, 4-[(4′-t-butylphenyl)methyl]-2-isopropyl-1-indene,
2,7-dimethyl-4-benzyl-1-indene, 2,7-dimethyl-4-cyclohexyl-1-indene, 2,7-dimethyl-4-(exo-2-norbornyl)-1-indene, 2-isopropyl-7-methyl-4-cyclohexyl-1-indene, 2-isopropyl-4-cyclohexyl-6-methyl-1-indene and 2-isopropyl-5-methyl-7-cyclohexyl-1-indene.

The 1-indenes of the formula (III) which are substituted by alkyl groups in the six-membered ring can be obtained in high yields and/or high purity by the process of the present invention. A particular advantage of the present invention is that readily obtainable and inexpensive chloroindene compounds of the formula (I) can be used as starting material, thus avoiding the necessity, as in the prior art, of using the corresponding bromine-substituted starting materials which are much more expensive. Contrary to the prevailing assumptions of the prior art that chlorine-substituted indenes do not display sufficient reactivity in the nickel-catalyzed cross-coupling reaction with aliphatic organometallic compounds to obtain economically sensible yields of the cross-coupling products, the process of the present invention has for the first time succeeded in achieving high product yields even when using the chloroindenes which are less reactive than the corresponding bromine compounds.

The present invention further provides indenes of the formula (IIa) or (IIb), 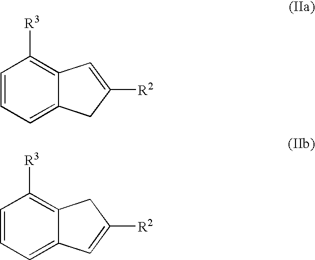
where
R² is C₁-C₁₀-alkyl and
R³ is a monocyclic or polycyclic C₅-C₁₅-alkyl group or a CH₂R⁴ group,
where
R⁴ is a C₆-C₁₄-aryl group, a C₇-C₁₅-alkylaryl group or a monocyclic or polycyclic C₅-C₁₅-alkyl group.

R² is a C₁-C₁₀-alkyl radical which may be linear, branched or cyclic. R² is preferably a C₁-C₆-alkyl radical such as methyl, ethyl, n-propyl, isopropyl or cyclohexyl.

Examples of preferred radicals R³ are cyclohexyl, 2′-adamantyl, exo-2-norbornyl, benzyl, methylbenzyl, (4′-isopropylphenyl)methyl, (4′-tert-butylphenyl)methyl, cyclohexylmethyl and 1′-adamantylmethyl, in particular benzyl or cyclohexyl.

The substituted indenes of the formula (II) prepared according to the present invention or the novel indenes of the formulae (IIa) and (IIb) can be used in all known applications of substituted indenes, for example as intermediates for the preparation of active compounds in the fields of crop protection, pharmacy, fine chemicals, liquid crystals and the like.

The substituted indenes of the formula (II) prepared according to the present invention or the novel indenes of the formulae (IIa) and (IIb) can be particularly advantageously used for the synthesis of metallocene complexes which are suitable for the polymerization of olefins, in particular α-olefins. The indenes of the formulae (II), (IIa) and (IIb) are particularly preferably used for the synthesis of chiral ansa-bisindenylmetallocenes.

The invention further provides ansa-bisindenylmetallocenes which are prepared from at least one indene of the formula (IIa) or (IIb) and in which the two indenyl ligands of the metallocene complex have different substituents in the 2 position.

The ansa-bisindenylmetallocenes of the present invention contain a metal of group 4, 5 or 6, in particular group 4, of the Periodic Table of the Elements, i.e. titanium, zirconium or hafnium. The metal is particularly preferably zirconium or hafnium, in particular zirconium.

The ansa-bisindenylmetallocenes of the present invention preferably bear different C₁-C₁₀-alkyl radicals in position 2 of the two indenyl radicals. In particular, the one alkyl radical in position 2 of the first indenyl ligand is a linear radical such as methyl, ethyl, n-propyl or n-butyl and the second alkyl radical in position 2 of the second indenyl ligand is an α-branched alkyl radical such as isopropyl, sec-butyl or cyclohexyl.

The preparation of such ansa-metallocene complexes using the indenes prepared according to the present invention or the indenes of the present invention is carried out by known, customary synthetic methods, for example by successive reaction of the indene (after deprotonation) with a bridging reagent such as dimethyldichlorosilane and suitable transition metal chlorides, for example zirconium tetrachloride or the like (again after deprotonation). Such synthetic routes are known from the prior art (WO 01/48034).

The invention is illustrated by the following examples which do not, however, restrict the scope of the invention:

General Procedures:

Preparation and handling of the organometallic compounds were carried out in the absence of air and moisture under argon (Schlenk technique or glove box). All solvents required were purged with argon and dried over molecular sieves before use. The ¹H-NMR spectra were measured in CDCl₃ and 400 MHz. The 7-chloro-2-alkyl-1-indanones and the 2-alkyl-4-aryl-1-indenes were prepared as described in WO 98/40331. The Grignard reagents used were prepared by standard methods as described in Holm, Torkil; J. Chem. Soc. Perkin Trans. 2; 1981, 464-467, or were purchased commercially (Aldrich).

### EXAMPLE 1

### 4-Chloro-2-methyl-1-indene

100 g (554 mmol) of 7-chloro-2-methyl-1-indanone and 13.5 g (358 mmol) of sodium borohydride together with 320 ml of toluene are placed under argon in a 2 l three-necked flask fitted with reflux condenser, dropping funnel, precision glass stirrer and internal thermometer at 50° C. 96 ml (2.38 mol) of methanol are added dropwise over a period of 30 minutes. After 3 hours at 50° C., the reaction is complete. After cooling to room temperature, 180 ml of water and then 280 ml of 10% strength sulfuric acid are added dropwise. The mixture is stirred vigorously for 30 minutes and is then transferred to a separating funnel. The phases are separated and the aqueous phase is extracted twice with 280 ml each time of toluene. The solvent is removed under reduced pressure. 450 ml of toluene and 0.8 g of p-toluenesulfonic acid are added to the residue and the mixture is boiled on a water separator until the reaction is complete (about 45 minutes). The organic phase is washed with 250 ml of a saturated sodium hydrogen carbonate solution and twice with 200 ml each time with water. After drying over magnesium sulfate, the solvent is removed under reduced pressure. The resulting crude product is fractionated in an oil pump vacuum to give the desired 4-chloro-2-methyl-1-indene in a yield of 84.7 g (515 mmol/93%).

### EXAMPLE 2

### 4-Chloro-2-ethyl-1-indene

100 g (514 mmol) of 7-chloro-2-ethyl-1-indanone and 12.6 g (334 mmol) of sodium borohydride together with 320 ml of toluene are placed under argon in a 2 l three-necked flask fitted with reflux condenser, dropping funnel, precision glass stirrer and internal thermometer at 50° C. 89 ml (2.20 mol) of methanol are added dropwise over a period of 30 minutes. After 3 hours at 50° C., the reaction is complete. After cooling to room temperature, 180 ml of water and then 280 ml of 10% strength sulfuric acid are added dropwise. The mixture is stirred vigorously for 30 minutes and is then transferred to a separating funnel. The phases are separated and the aqueous phase is extracted twice with 280 ml each time of toluene. The solvent is removed under reduced pressure. 450 ml of toluene and 0.8 g of p-toluenesulfonic acid are added to the residue and the mixture is boiled on a water separator until the reaction is complete (about 45 minutes). The organic phase is washed with 250 ml of a saturated sodium hydrogen carbonate solution and twice with 200 ml each time with water. After drying over magnesium sulfate, the solvent is removed under reduced pressure. The resulting crude product is fractionated in an oil pump vacuum to give the desired 4-chloro-2-ethyl-1-indene in a yield of 82.4 g (461 mmol/90%).

### EXAMPLE 3

### 4-Chloro-2-isopropyl-1-indene

100 g (479 mmol) of 7-chloro-2-isopropyl-1-indanone and 11.8 g (311 mmol) of sodium borohydride together with 320 ml of toluene are placed under argon in a 2 l three-necked flask fitted with reflux condenser, dropping funnel, precision glass stirrer and internal thermometer at 50° C. 83 ml (2.06 mol) of methanol are added dropwise over a period of 30 minutes. After 3 hours at 50° C., the reaction is complete. After cooling to room temperature, 180 ml of water and then 280 ml of 10% strength sulfuric acid are added dropwise. The mixture is stirred vigorously for 30 minutes and is then transferred to a separating funnel. The phases are separated and the aqueous phase is extracted twice with 280 ml each time of toluene. The solvent is removed under reduced pressure. 450 ml of toluene and 0.8 g of p-toluenesulfonic acid are added to the residue and the mixture is boiled on a water separator until the reaction is complete (about 45 minutes). The organic phase is washed with 250 ml of a saturated sodium hydrogen carbonate solution and twice with 200 ml each time with water. After drying over magnesium sulfate, the solvent is removed under reduced pressure. The resulting crude product is fractionated in an oil pump vacuum to give the desired 4-chloro-2-isopropyl-1-indene in a yield of 83.6 g (434 mmol/91%).

### EXAMPLE 4

### 2,4-Dimethyl-1-indene

10 g (60.7 mmol) of 4-chloro-2-methyl-1-indene together with 30 ml of diethyl ether are placed under argon in a 500 ml four-necked flask. 0.65 g (1.2 mmol, 2 mol %) of 1,3-bis(diphenylphosphino)propanenickel(II) chloride are added. After cooling to 0° C., 101 ml of methylmagnesium bromide (0.304 mol, 3 M in diethyl ether) are added dropwise at such a rate that the temperature does not exceed 5° C. (about 20 minutes). After addition is complete, the ice bath is removed and the mixture is stirred at room temperature for 1 hour. The green suspension is then refluxed until the reaction is complete (12 h). The reaction mixture is carefully poured onto 125 g of ice and acidified with 20 ml of concentrated HCl. After separation of the phases, the organic phase is extracted twice with 50 ml each time of dichloromethane. The combined organic phases are dried over magnesium sulfate and the solvent is removed under reduced pressure to give the product in a yield of 8.5 g (58.8 mmol, 97%) and a purity of 99% (GC).

### EXAMPLE 5

### 4-Cyclohexyl-2-methyl-1-indene

10 g (60.7 mmol) of 4-chloro-2-methyl-1-indene together with 30 ml of diethyl ether are placed under argon in a 500 ml four-necked flask. 0.65 g (1.2 mmol, 2 mol %) of 1,3-bis(diphenylphosphino)propanenickel(II) chloride are added. After cooling to 0° C., 91 ml (91 mmol/1 M in diethyl ether) of cyclohexylmagnesium bromide are added dropwise at such a rate that the temperature does not exceed 5° C. (about 20 minutes). After addition is complete, the ice bath is removed and the mixture is stirred at room temperature for 1 hour. The green suspension is then refluxed until the reaction is complete (12 h). The reaction mixture is carefully poured onto 125 g of ice and acidified with 20 ml of concentrated HCl. After separation of the phases, the organic phase is extracted twice with 50 ml each time of dichloromethane. The combined organic phases are dried over magnesium sulfate and the solvent is removed under reduced pressure to give the product in a yield of 12.7 g (60.0 mmol, 99%) and a purity of 99% (GC).

### EXAMPLE 6

### 4-Cyclohexylmethyl-2-methyl-1-indene

5 g (30 mmol) of 4-chloro-2-methyl-1-indene together with 15 ml of diethyl ether are placed under argon in a 250 ml four-necked flask. 0.33 g (0.6 mmol, 2 mol %) of 1,3-bis(diphenylphosphino)propanenickel(II) chloride are added. After cooling to 0° C., 45 ml (45 mmol/1 M in diethyl ether) of cyclohexylmethylmagnesium bromide are added dropwise at such a rate that the temperature does not exceed 5° C. (about 20 minutes). After addition is complete, the ice bath is removed and the mixture is stirred at room temperature for 1 hour. The green suspension is then refluxed until the reaction is complete (12 h). The reaction mixture is carefully poured onto 60 g of ice and acidified with 10 ml of concentrated HCl. After separation of the phases, the organic phase is extracted twice with 50 ml each time of dichloromethane. The combined organic phases are dried over magnesium sulfate and the solvent is removed under reduced pressure to give the product in a yield of 6.7 g (29.7 mmol, 99%) and a purity of 99% (GC).

### EXAMPLE 7

### 4-Benzyl-2-methyl-1-indene

5 g (30 mmol) of 4-chloro-2-methyl-1-indene together with 15 ml of diethyl ether are placed under argon in a 250 ml four-necked flask. 0.33 g (0.6 mmol, 2 mol %) of 1,3-bis(diphenylphosphino)propanenickel(II) chloride are added. After cooling to 0° C., 45 ml (45 mmol/1 M in diethyl ether) of benzylmagnesium chloride are added dropwise at such a rate that the temperature does not exceed 5° C. (about 20 minutes). After addition is complete, the ice bath is removed and the mixture is stirred at room temperature for 1 hour. The green suspension is then refluxed (48 h). The reaction mixture is carefully poured onto 60 g of ice and acidified with 10 ml of concentrated HCl. After separation of the phases, the organic phase is extracted twice with 50 ml each time of dichloromethane. The combined organic phases are dried over magnesium sulfate and the solvent is removed under reduced pressure. The resulting yellow oil is stored in a refrigerator overnight, resulting in crystallization of the product. The crystals obtained in this way are isolated by filtration and washing with a little cold methanol, giving the product in the form of white crystals in a yield of 1.44 g (6.5 mmol, 22%) and a purity of 99% (GC).

### EXAMPLE 8

### 4-Cyclohexyl-2-ethyl-1-indene

10 g (56 mmol) of 4-chloro-2-ethyl-1-indene together with 30 ml of diethyl ether are placed under argon in a 500 ml four-necked flask. 0.61 g (1.1 mmol, 2 mol %) of 1,3-bis(diphenylphosphino)propanenickel(II) chloride are added. After cooling to 0° C., 77 ml (77 mmol/1 M in diethyl ether) of cyclohexylmagnesium bromide are added dropwise at such a rate that the temperature does not exceed 5° C. (about 20 minutes). After addition is complete, the ice bath is removed and the mixture is stirred at room temperature for 1 hour. The green suspension is then refluxed until the reaction is complete (12 h). The reaction mixture is carefully poured onto 125 g of ice and acidified with 20 ml of concentrated HCl. After separation of the phases, the organic phase is extracted twice with 50 ml each time of dichloromethane. The combined organic phases are dried over magnesium sulfate and the solvent is removed under reduced pressure to give the product in a yield of 12.1 g (53 mmol, 96%) and a purity of 99% (GC).

### EXAMPLE 9

### 4-Cyclohexyl-2-isopropyl-1-indene

10 g (52 mmol) of 2-chloro-2-isopropyl-1-indene together with 30 ml of diethyl ether are placed under argon in a 500 ml four-necked flask. 0.56 g (1 mmol, 2 mol %) of 1,3-bis(diphenylphosphino)propanenickel(II) chloride are added. After cooling to 0° C., 77 ml (77 mmol/1 M in diethyl ether) of cyclohexylmagnesium bromide are added dropwise at such a rate that the temperature does not exceed 5° C. (about 20 minutes). After addition is complete, the ice bath is removed and the mixture is stirred at room temperature for 1 hour. The green suspension is then refluxed until the reaction is complete (12 h). The reaction mixture is carefully poured onto 125 g of ice and acidified with 20 ml of concentrated HCl. After separation of the phases, the organic phase is extracted twice with 50 ml each time of dichloromethane. The combined organic phases are dried over magnesium sulfate and the solvent is removed under reduced pressure to give the product in a yield of 11.3 g (47 mmol, 90%) and a purity of 96% (GC).

### EXAMPLE 10

### Dimethylsilanediylbis(2-methyl-4-cyclohexylindenyl)zirconium dichloride

3.0 g (14.1 mmol) of 4-cyclohexyl-2-methyl-1-indene together with 20 ml of toluene/3 ml of THF are placed in a 100 ml four-necked flask. The solution is admixed at room temperature with 6.0 ml (15.1 mmol) of n-BuLi (2.5 M in toluene) and subsequently stirred at 80° C. for 1 hour. After cooling to 40° C., 0.85 ml (7.1 mmol) of dimethyldichlorosilane are added quickly. The mixture is subsequently stirred for another 3 hours at 60° C. 5.6 ml (14.1 mmol) of n-BuLi (2.5 M in toluene) are added to the gray suspension at room temperature and the mixture is stirred at 80° C. for another 1 hour. After cooling to room temperature, 1.8 g (7.7 mmol) of zirconium tetrachloride are added a little at a time and the mixture is stirred at 45° C. for 2 hours. The solvent is removed under reduced pressure. The orange residue is admixed with 3 g of Celite and 40 ml of toluene, stirred at 50° C. for 30 minutes and then filtered through a G3 Frit to remove the lithium chloride. The solid on the filter is washed with 30 ml of hot toluene and the filtrate is evaporated to 10 ml under reduced pressure. At room temperature, a solid precipitates and is removed by filtration. The filtrate is stored in a refrigerator for 12 hours and the orange crystals are isolated by filtration through a G3 Frit and washing with 5 ml of cold toluene. Yield: 0.7 g (15%), rac/meso=5.6:1.

### EXAMPLE 11

### Dimethylsilanediylbis(2-methyl-4-cyclohexylmethylindenyl)zirconium dichloride

4.0 g (17.7 mmol) of 4-cyclohexylmethyl-2-methyl-1-indene together with 40 ml of toluene/4 ml of THF are placed in a 100 ml four-necked flask. The solution is admixed at room temperature with 7.6 ml (18.9 mmol) of n-BuLi (2.5 M in toluene) and subsequently stirred at 80° C. for 1 hour. After cooling to 40° C., 1.06 ml (8.8 mmol) of dimethyldichlorosilane are added quickly. The mixture is subsequently stirred for another 3 hours at 60° C. 7.0 ml (17.7 mmol) of n-BuLi (2.5 M in toluene) are added to the brown suspension at room temperature and the mixture is stirred at 80° C. for another 1 hour. After cooling to room temperature, 2.26 g (9.7 mmol) of zirconium tetrachloride are added a little at a time and the mixture is stirred at 45° C. for 2 hours. The solvent is removed under reduced pressure. The orange residue is admixed with 3 g of Celite® and 40 ml of toluene, stirred at 50° C. for 30 minutes and then filtered through a G3 Frit to remove the lithium chloride. The solid on the filter is washed with 30 ml of hot toluene and the filtrate is evaporated to 10 ml under reduced pressure. Fractional crystallization from a heptane/toluene mixture gives the product in the form of a powder in a yield of 1.6 g (2.6 mmol, 30%), rac/meso=1.1:1.

### EXAMPLE 12

### (4-Cyclohexyl-2-methyl-1H-inden-1-yl)dimethylchlorosilane

16.1 g (76 mmol) of 4-cyclohexyl-2-methyl-1-indene together with 160 ml of toluene and 5 ml of DME are placed in a reaction vessel. 28.4 ml (76 mmol) of a butyllithium solution are added dropwise to the above solution, and the mixture is stirred at 80° C. for 1 hour after the addition is complete. The reaction solution is slowly added dropwise to a solution of 27.7 ml (229 mmol) of dimethyldichlorosilane in 260 ml of THF which has been precooled to −40° C. After warming to room temperature, the reaction mixture is stirred overnight. The solvent is subsequently removed in an oil pump vacuum and the residue which remains is taken up in 100 ml of toluene. The insoluble lithium chloride is separated off via a G4 Frit and the solvent is removed from the filtrate in an oil pump vacuum. The product is obtained in a yield of 23.1 g (76 mmol, 100%) as a highly viscous oil.

### EXAMPLE 13

### Dimethylsilanediyl(2-methyl-4-cyclohexylindenyl)(2-isopropyl-4-cyclohexylindenyl)zirconium dichlorde

3.3 ml (8.3 mmol) of n-butyllithium (2.5 M in toluene) are added at room temperature to a solution of 2 g (8.3 mmol) of 4-cyclohexyl-2-isopropyl-1-indene in 20 ml of toluene/2 ml of THF. The solution is stirred at 80° C. for 1 hour. After cooling to room temperature, 2.53 g (8.3 mmol) of (4-cyclohexyl-2-methyl-1H-inden-1-yl)dimethylchlorosilane are added and the mixture is stirred at 50° C. for 3 hours. After cooling to room temperature, 6.6 ml (16.6 mmol) of n-butyllithium (2.5 M in toluene) are added and the mixture is stirred at 80° C. for 1 hour. After cooling to room temperature, 1.93 g (8.3 mmol) of zirconium tetrachloride are added. The mixture is stirred at 45° C. for another 2 hours and is then stirred overnight at room temperature. The solvent is removed under reduced pressure. The orange residue is admixed with 6 g of Celite® and 80 ml of toluene, stirred at 50° C. for 30 minutes and then filtered through a G3 Frit to remove the lithium chloride. The solid on the filter is washed with 75 ml of hot toluene and the filtrate is evaporated to 10 ml under reduced pressure. Fractional crystallization from a heptane/toluene mixture gives the product in the form of an orange powder in a yield of 1.3 g (1.9 mmol, 23%), rac/meso=4:1.

### EXAMPLE 14

### Dimethylsilanediyl(2-methyl-4-cyclohexylmethylindenyl)(2-isopropyl-4-(4′-tbutylphenyl)indenyl)zirconium dichloride

3.5 ml (8.8 mmol) of n-butyllithium (2.5 M in toluene) are added at room temperature to a solution of 2 g (8.8 mmol) of 4-cyclohexylmethyl-2-isopropyl-1-indene in 20 ml of toluene/2 ml of THF. The solution is stirred at 80° C. for 1 hour. After cooling to room temperature, 3.38 g (8.8 mmol) of 2-isopropyl-4-(4′-t-butylphenyl)indenyidimethylchlorosilane are added and the mixture is stirred at 50° C. for 3 hours. After cooling to room temperature, 7.0 ml (17.7 mmol) of n-butyllithium (2.5 M in toluene) are added. The mixture is stirred at 45° C. for another 2 hours and is then stirred overnight at room temperature. The solvent is removed under reduced pressure. The orange residue is admixed with 6 g of Celite® and 80 ml of toluene, stirred at 50° C. for 30 minutes and then filtered through a G3 Frit to remove the lithium chloride. The solid on the filter is washed with 75 ml of hot toluene and the filtrate is evaporated to 14 ml under reduced pressure. Fractional crystallization from a heptane/toluene mixture gives the product in the form of an orange powder in a yield of 1.6 g (2.2 mmol, 25%), rac/meso=3:1.

We claim:

1. A process for preparing indenes of the formula (II) from indenes of the formula (I) by reaction with compounds of the formula (III) 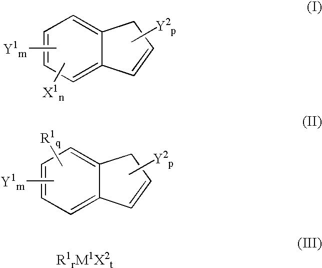
where
X¹ is chlorine;
Y¹ are identical or different and are selected independently from the group consisting of C₁-C₄₀ groups, e.g. C₁-C₂₅-alkyl, C₂-C₂₅-alkenyl, C₂-C₂₅-alkynyl, C₃-C₁₅-alkylalkenyl, C₃-C₁₅-alkylalkynyl, C₆-C₂₄-aryl, C₄-C₂₄-heteroaryl, C₅-C₂₄-alkylheteroaryl, C₇-C₃₀-arylalkyl, C₇-C₃₀-alkylaryl, C₁-C₁₂-alkoxy, C₆-C₂₄-aryloxy, fluorinated C₁-C₂₅-alkyl, fluorinated C₆-C₂₄-aryl, fluorinated C₇-C₃-arylalkyl, fluorinated C₇-C₃₀-alkylaryl, and the fluorine atom and heteroatom-containing groups, e.g. boron-, silicon-, nitrogen-, oxygen- or sulfur-containing groups, which may bear one or more substituents, where a plurality of groups Y¹ may also together form a cyclic aliphatic or aromatic ring system which may in turn be substituted and may contain heteroatoms;
Y² are identical or different and are selected independently from the group consisting of C₁-C₄₀ groups, e.g. C₁-C₂₅-alkyl, C₂-C₂₅-alkenyl, C₂-C₂₅-alkynyl, C₃-C₁₅-alkylalkenyl, C₃-C₁₅-alkylalkynyl, C₆-C₂₄-aryl, C₄-C₂₄-heteroaryl, C₅-C₂₄-alkylheteroaryl, C₇-C₃₀-arylalkyl, C₇-C₃₀-alkylaryl, C₁-C₁₂-alkoxy, C₆-C₂₄-aryloxy, fluorinated C₁-C₂₅-alkyl, fluorinated C₆-C₂₄-aryl, fluorinated C₇-C₃₀-arylalkyl, fluorinated C₇-C₃₀-alkylaryl, and heteroatom-containing groups, e.g. boron-, silicon-, nitrogen-, oxygen- or sulfur-containing groups, which may bear one or more substituents, where a plurality of groups y² may also together form a cyclic aliphatic or aromatic ring system which may in turn be substituted and may contain heteroatoms;
R¹ are identical or different and are selected independently from the group consisting of linear, branched or cyclic aliphatic hydrocarbon groups, e.g. C₁-C₂₅-alkyl which may in turn bear a variety of substituents, and groups bound via an aliphatic group to the indenyl skeleton, e.g. C₃-C₁₅-alkenylalkyl, C₃-C₁₅-alkynylalkyl, C₅-C₂₄-heteroarylalkyl, C₇-C₃₀-arylalkyl, C₂-C₃alkyloxyalkyl, C₇-C₃₀-aryloxyalkyl, C₈-C₃₀-alkylarylalkyl, and other heteroatom-containing groups which are bound via an aliphatic group to the indenyl skeleton, e.g. boron-, silicon-, nitrogen-, oxygen- or sulfur-containing groups, and may bear one or more substituents;
M¹ is an element of group 1, 2, 12, 13 or 14 of the Periodic Table of the Elements;
X² are identical or different and are selected independently from the group consisting of halogen atoms, the hydroxy group, alkoxy groups, aryloxy groups, mesylate, tosylate and triflate;
m is an integer from 0 to 3;
n is an integer from 1 to 4;
p is an integer from 0 to 4;
q is an integer from 1 to 4;
r is 1, 2 or 3, and
t is 0, 1 or 2, where r+t corresponds to the oxidation number of M¹;
wherein the indenes of the formula (I) are reacted with appropriate aliphatic organometallic compounds of the formula (III) in the presence of a transition metal catalyst.

2. A process as claimed in claim 1, wherein
M¹ is Li, Mg, B or Zn, and
X² are identical or different and are selected independently from the group consisting of halogen atoms, the hydroxy group, alkoxy groups and aryloxy groups.

3. A process as claimed in claim 1, wherein at least one transition metal catalysts selected from the group consisting of nickel(II) acetylacetonate, [1,2-bis(diphenylphosphino)ethane]nickel(II) chloride, [1,3-bis(diphenylphosphino)propane]nickel(II)chloride, [1,1′-bis(diphenylphosphino)ferrocene]nickel(II) chloride, bis(tributylphosphine)nickel(II) bromide, bis(tributylphosphine)nickel(II) chloride, bis(triphenylphosphine)nickel(II) chloride, bis(triphenylphosphine)dicarbonylnickel(0), [1,2-bis(dimethylphosphino)ethane]nickel(II) chloride, bis(triethylphosphine)nickel(II) chloride, bis(triphenylphosphine)palladium(II) chloride, tetrakis(triphenylphosphine)palladium(0), [1,2-bis(diphenylphosphino)ethane]palladium(II) chloride and the [1,1′-bis(diphenylphosphino)ferrocene]nickel(II) chloride-methylene chloride complex is used.

4. A process as claimed in claim 1, wherein the transition metal catalyst used is [1,3-bis(diphenylphosphino)propane]nickel(II) chloride.

5. A process as claimed in claim 1 any of claims 14, wherein the transransition metal catalyst is added in an amount of from 0.01 to 5 mol %, based on chloroindene of the formula (I) used.

6. A process as claimed in claim 1, wherein the chloroindenes of the formula (I) which are used are selected from the groups consisting of: 4-chloro-1-indene; 5-chloro-1-indene; 6-chloro-1-indene; 7-chloro-1-indene; 2-methyl-4-chloro-1-indene; 2,7-dimethyl-4-chloro-1-indene; 2,4-dimethyl-7-chloro-1-indene; 2-methyl-5-chloro-1-indene; 2-methyl-6-chloro-1-indene; 2-methyl-7-chloro-1-indene; 2-ethyl-4-chloro-1-indene; 2-ethyl-5-chloro-1-indene; 2-ethyl-6-chloro-1-indene; 2-ethyl-7-chloro-1-indene; 2-propyl-4-chloro-1-indene; 2-propyl-5-chloro-1-indene; 2-propyl-6-chloro-1-indene; 2-propyl-7-chloro-1-indene; 2-i-propyl-4-chloro-1-indene; 2-i-propyl-5-chloro-1-indene; 2-i-propyl-6-chloro-1-indene; 2-i-propyl-7-chloro-1-indene; 2-butyl-4-chloro-1-indene; 2-butyl-5-chloro-1-indene; 2-butyl-6-chloro-1-indene; 2-butyl-7-chloro-1-indene; 2-s-butyl-4-chloro-1-indene; 2-s-butyl-5-chloro-1-indene; 2-s-butyl-6-chloro-1-indene; 2-s-butyl-7-chloro-1-indene; 2-t-butyl-4-chloro-1-indene; 2-t-butyl-5-chloro-1-indene; 2-t-butyl-6-chloroindene; 2-t-butyl-7-chloroindene.

7. An indene of the fromula (IIa) or (IIb), 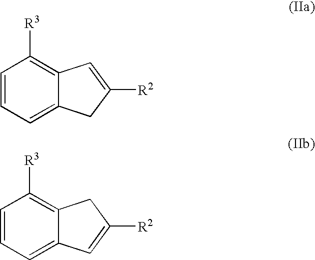
where
R² is C₁-C₁₀-alkyl and
R³ is a monocyclic or polycyclic C₅-C₁₅-alkyl group or a CH₂R⁴ group,
where
R⁴ is a C₆-C₁₄-aryl group, a C₇-C₁₅-alkylaryl group or a monocyclic or polycyclic C₅-C₁₅-alkyl group.

8. A process for the synthesis of metallocene compounds, said process comprising synthesizing the compounds from indenes of the formula (II) obtained as set forth in claim 1.

9. An ansa-bisindenylmetallocene prepared from at least one indene of the formula (IIa) or (IIb) as claimed in claim 7, wherein the two indenyl ligands of the metallocene have different substituents in the 2 position.

10. A process for preparing olefins, said process comprising polymerizing said olefins in the presence of at least one ansa-bisindenylmetallocene prepared from at least one indene of the formula (II) as set forth in claim 1.
